(19) United States
(12) Patent Application Publication
(10) Pub. No.: US 2009/0088716 A1
(43) Pub. Date: Apr. 2, 2009

(54) Article for absorption of human exudate

(76) Inventor: Oriyomi Nwokeji, Succasunna, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 11/906,199

(22) Filed: Oct. 1, 2007

(51) Int. Cl. A61F 13/511; A61F 13/476; A61F 13/56

(52) U.S. Cl. 604/385.04; 604/385.08; 604/386

(57) ABSTRACT

The invention encompasses an absorbent article having a shape so as to guard against leakage, especially when the wearer is in a prone or supine position. In one embodiment, the absorbent article comprises a body, including an exudate impermeable backsheet, and exudate permeable topsheet that is attached to the bottom sheet, and an absorbent core that is positioned between the backsheet and topsheet. Further, a front end comprises a first upraised peak, while a back end comprises a second upraised peak, with a central portion extending between the first and second upraised peaks.

### BACKGROUND OF THE INVENTION

There are known a number of devices designed to absorb and trap various body fluids that are discharged from the urogenital and gastrointestinal tracts, such as urine, menses and fecal matter. Some of these products are designed to be worn externally (e.g., sanitary napkins, diapers), while others are worn internally (e.g., tampons).

The goal of such devices is to contain the aforementioned fluids. However, many of these devices, especially the externally worn devices, have a tendency to leak from the sides, and from the front or back. The potential for leakage can be exacerbated when the wearer is in a prone or supine position. Accordingly, there is a need for such a device that exhibits an improved ability to guard against leakage.

### SUMMARY OF THE INVENTION

The invention encompasses an absorbent article having a shape so as to guard against leakage, especially when the wearer is in a prone or supine position. In one embodiment, the absorbent article comprises a body, including an exudate impermeable backsheet, and exudate permeable topsheet that is attached to the bottom sheet, and an absorbent core that is positioned between the backsheet and topsheet. Further, a front end comprises a first upraised peak, while a back end comprises a second upraised peak, with a central portion extending between the first and second upraised peaks.

An absorbent article for absorption of human exudate, comprising a body including an exudate impermeable backsheet an exudate permeable topsheet attached to the backsheet and an absorbent core positioned between the topsheet and backsheet, wherein said body includes a front end comprising a first upraised peak a back end comprising a second upraised peak and a central portion extending between said first and second upraised peaks, said center portion having a height less than a height of said first and second upraised peaks.

An absorbent article for use on the body of a wearer, comprising a body having a longitudinal axis, said body including an exudate impermeable backsheet an exudate permeable topsheet attached to the backsheet and an absorbent core positioned between the topsheet and backsheet, wherein said body includes a front end comprising a first upraised peak a back end comprising a second upraised peak a central portion extending between said first and second upraised peaks, said center portion having a height less than a height of said first and second upraised peaks.

An absorbent article for absorption of human exudate, comprising a body having a longitudinal axis, including an exudate impermeable backsheet an exudate permeable topsheet attached to the backsheet and an absorbent core positioned between the topsheet and backsheet, wherein said body exhibits undulation when viewed in a cross-section taken along said longitudinal axis.

### BRIEF DESCRIPTION OF THE DRAWINGS

FIG. 1 is a top plan view of a sanitary napkin according to an embodiment of the present invention.

FIG. 2 is a front view of the sanitary napkin of FIG. 1.

FIG. 3 is a rear view of the sanitary napkin of FIG. 1.

FIG. 4 is a perspective view of the sanitary napkin of FIG. 1.

FIG. 5 is a side view of the sanitary napkin of FIG. 1.

FIG. 6 is a front view of a wearer using the sanitary napkin of FIG. 1.

FIG. 7 is a rear view of a wearer using the sanitary napkin of FIG. 1.

FIG. 8 is a side view of a wearer using the sanitary napkin of FIG. 1.

FIG. 9 is a perspective view of a sanitary napkin according to another embodiment of the present invention.

### DETAILED DESCRIPTION

The invention is directed to a device intended to absorb and contain body exudate, and prevent leakage of same, regardless of a wearer's position. It is especially useful in preventing leakage of exudate from a front or back of the wearer, particularly if the user is in a supine position.

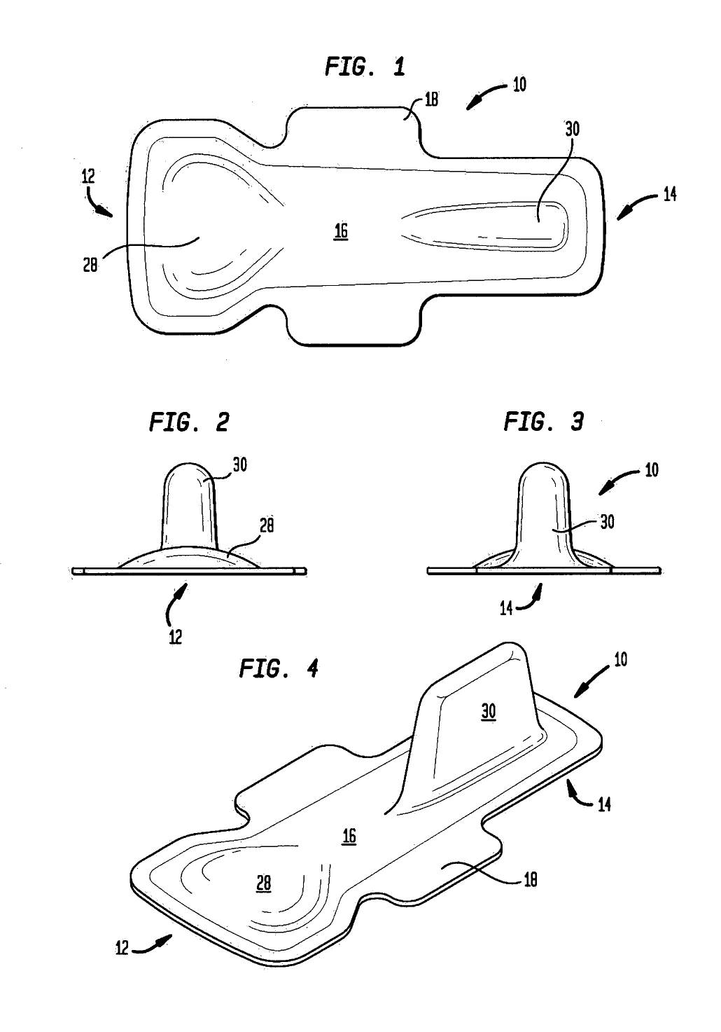

FIGS. 1-5 illustrate one embodiment of the invention in the nature of a sanitary napkin 10 generally in the form of an elongated body having a first end 12 and a second end 14. Between the first and second ends 12, 14, respectively, is a center 16. On either side of the center 16 are optional wings 18.

The wings 18 provide protection against leakage of body fluids in a direction perpendicular to the longitudinal axis of the sanitary napkin 10. The wings 18 can be coplanar and extend outwardly from the sanitary napkin 10. The wings 18 can have a variety of different shapes, such as for example, semi-circular, bell shape, rectangular, etc. The wings 18 can have an adhesive or the like for affixing to a wearer's undergarments.

Further, the sanitary napkin 10 includes a topsheet 20 and a backsheet 22. The topsheet 20 and backsheet 22 are joined at their respective perimeters to form a protruding edge 24 around the sanitary napkin 10. The wings 18 may be formed of the topsheet 20 and the backsheet 22 or be formed of additional material that is then attached to the sanitary napkin 10. Encased between the topsheet 20 and the backsheet 22 is a center layer 26.

The topsheet 20 is generally permeable and porous, allowing exudate to travel through it to the center layer 26. However, the backsheet 22 is generally impermeable, preventing exudate from traveling from the center layer 26 through the backsheet 22 to the wearer. The topsheet 20 can be formed of a natural material such as cotton, a polymer, or a like material having a porous or perforated nature. The backsheet 22 can be formed of a polymer, natural materials or a like material that is generally impermeable. The center layer 26 is formed of a highly absorbent material such as a sponge, foam, or any material that is known to have highly absorbent tendencies. The center layer 26 may include one or more layers of the same or different types of absorbent material.

Further, the sanitary napkin 10 can have an adhesive layer 27 for affixing the sanitary napkin 10 to a wearer's undergarments. The adhesive layer 27 can be formed of a single strip of adhesive or several strips of adhesive. The adhesive layer 27 may also be formed on the back of the wings 18 to affix the wings 18 to the undergarment.

The first and second ends 12, 14 of the sanitary napkin 10 include upraised peaks, 28 and 30, respectively, formed of the center layer 26. The peaks 28 and 30 are defined as having a volume of mass located at point where the volume of mass rises above the remainder of the sanitary napkin 10. The peaks may be formed of additional layers of material or with one layer of material that is amassed together to form the peak. The peaks 28 and 30 have sides that form the slopes for the sanitary napkin 10.

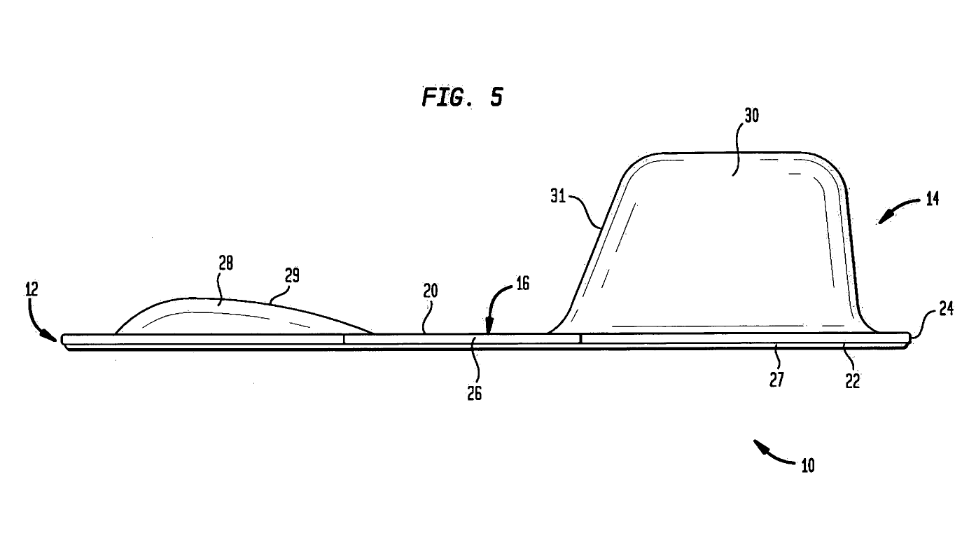

The peaks 28, 30 may be equal in height or vary in height from one another. The peaks 28, 30 may also have different shapes and profiles. Preferably, the first peak 28 has a smaller height than the second peak 30. Further, as best seen in FIG. 5, the first peak 28 has a first peak slope 29 that extends from the top of the peak 28 to the center 16. The first peak slope 29 has a gradual slope towards the center 16. Further, the first peak slope 29 is wide adjacent the first end 12 and narrow toward the center 16. The first peak slope 29 may generally fan out from the center toward the first end and can be in the form of a V or U, wherein the legs approach the first end 12 from the center 16. Such a shape covers substantially all of the front pudendal region of the wearer to prevent exudate from escaping from the sanitary napkin 10. The second peak 30 has a second peak slope 31 that extends from the top of the peak 30 to the center 16. The second peak slope 31 has a sharper slope than the first peak slope 29. Further, the center 16 can also have a gradual slope as it extends from a direction near the first end 12 to a direction near the second end 14. Alternately, the center can be flat, of uniform thickness.

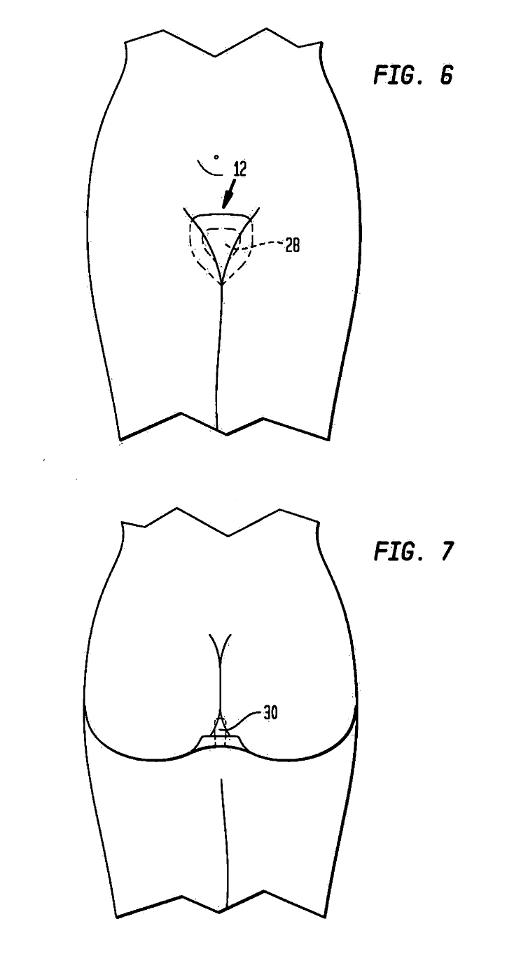

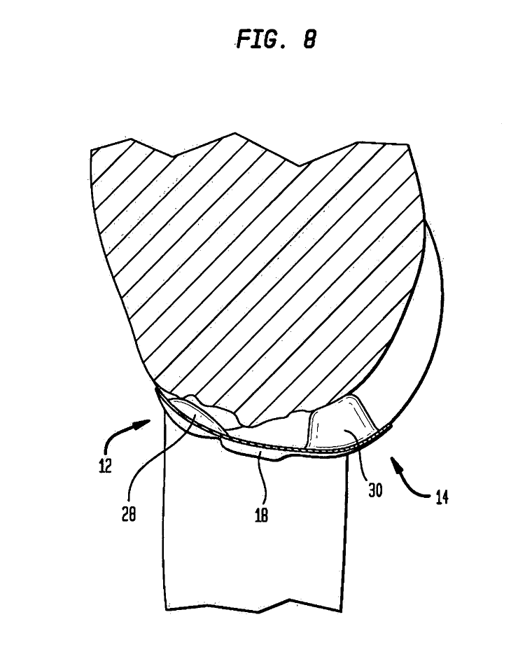

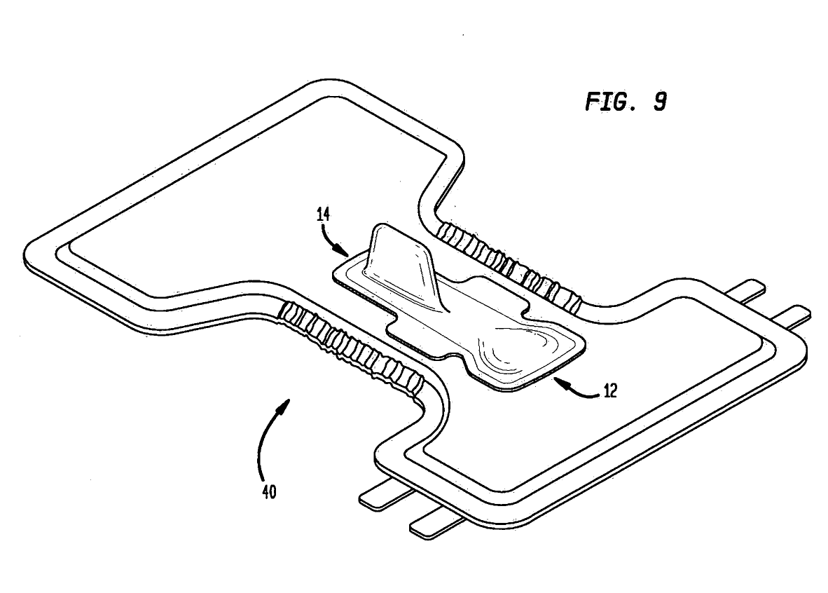

The gradual slope of the first peak slope 29 is preferred because of the generally gradual slope of the pudendal region of the wearer. Preferably, the gradual slope can be defined by any slope greater than one. The second peak slope 31 preferably includes a sharp slope to accommodate the anal region of the wearer. As seen in FIGS. 6-8, in an embodiment of the present invention, the second slope 31 disappears into the buttocks of the wearer such that, from a side view of a wearer using the sanitary napkin 10, the second slope 31 is not visible. Thus, the second slope 31 fills the gap formed by the buttocks, unlike conventional sanitary napkins. By filling this gap, the sanitary napkin 10 of the present invention, prevents exudate from escaping through this gap. Preferably, the sharp slope can be defined as having a slope of less than 1. The slopes of the first and second ends 12, 14 prevent any exudate from flowing out of the sanitary napkin 10, particularly when the wearer is in a supine position.

For a sanitary napkin 10 appropriate for a small/medium woman, the sanitary napkin 10 generally has a length approximating 12 inches. For such a small/medium size sanitary napkin 10, the first and second ends 12, 14 can have a height of about 1 inch. The center 16 can have a thickness of half an inch or less. Generally, the length of the first and second peaks 28, 30 are about 3 inches. These dimensions can vary according to the size of the sanitary napkin 10 and the size of the wearer. Therefore sizes described here are only exemplary.

In an embodiment of the present invention, the first and second ends 12, 14 can be of equal width or varying widths. Preferably, the first end 12 is wider than the second end 14. As shown in FIG. 6, the wider first end 12 covers the pudendal region of the wearer. Further, the first and second ends 12, 14 can be of equal width or varying width than the center 16. Preferably, the first and second ends 12, 14 are wider than the center 16. The width of the wings 18 can be larger than either or both of the first or second ends 12, 14 or be of the same width as the first or second ends 12, 14. Generally, the wings are wider than the first or second ends, 12, 14 or center 16. For a small/medium size sanitary napkin 10, the first end 12 can have a width of about five inches. The second end 14 can have a width of about 3 inches. As stated previously, these dimensions can vary according to the size of the sanitary napkin 10 and the size of the wearer. The larger width of the first end 12 prevents any exudate from escaping the sanitary napkin 10 when the wearer is in a supine position. Also, the smaller width of the second end 14 provides comfort to the user because of the narrow area between the buttocks.

In another embodiment of the invention shown in FIG. 9, the described device having upraised peaks can be incorporated for an incontinence pad 40 for adults, diapers for children or the like and are not limited to feminine hygiene napkins for women.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An absorbent article for absorption of human exudate, comprising:
a body including;
an exudate impermeable backsheet;
an exudate permeable topsheet attached to the backsheet; and
an absorbent core positioned between the topsheet and backsheet, wherein said body includes:
a front end comprising a first upraised peak;
a back end comprising a second upraised peak; and
a central portion extending between said first and second upraised peaks, said center portion having a height less than a height of said first and second upraised peaks.

2. The absorbent article of claim 1, wherein said body includes two lateral sides.

3. The absorbent article of claim 2, further comprising wings extending perpendicular from the lateral sides.

4. The absorbent article of claim 2, wherein said wings are positioned perpendicular to the lateral sides of the central portion.

5. The absorbent article of claim 1, wherein said first upraised peak rises sharply from the front end, and gradually declines towards the center portion.

6. The absorbent article of claim 1, wherein said second upraised peak rises sharply from the back end, and sharply declines towards the center portion.

7. The absorbent article of claim 1, wherein said first and second upraised peaks are of equal height.

8. The absorbent article of claim 1, wherein said first upraised peak is shorter than said second upraised peak.

9. The absorbent article of claim 1, wherein said first upraised peak is higher than said second upraised peak.

10. The absorbent article of claim 1, wherein said first upraised peak has a first slope greater than 1.

11. The absorbent article of claim 1, wherein said second upraised peak has a second slope less than 1.

12. The absorbent article of claim 1, further comprising a means for maintaining said absorbent article in operable association with the wearer.

13. An absorbent article for use on the body of a wearer, comprising:
a body having a longitudinal axis, said body including:
an exudate impermeable backsheet;
an exudate permeable topsheet attached to the backsheet; and
an absorbent core positioned between the topsheet and backsheet, wherein said body includes:
a front end comprising a first upraised peak;
a back end comprising a second upraised peak;
a central portion extending between said first and second upraised peaks, said center portion having a height less than a height of said first and second upraised peaks.

14. The absorbent article of claim 13, further comprising a means for maintaining said absorbent article in operable association with the wearer.

15. The absorbent article of claim 14, wherein the means for maintaining said absorbent article in operable association with the wearer comprises an adhesive.

16. An absorbent article for absorption of human exudate, comprising:
a body having a longitudinal axis, including:
an exudate impermeable backsheet;
an exudate permeable topsheet attached to the backsheet; and
an absorbent core positioned between the topsheet and backsheet, wherein said body exhibits undulation when viewed in a cross-section taken along said longitudinal axis.

17. The absorbent article of claim 16, wherein said body includes a front end, a back end, a center portion, and two lateral sides.

18. The absorbent article of claim 17, further comprising wings positioned perpendicular to the lateral sides.

19. The absorbent article of claim 17, wherein said wings are positioned perpendicular to the lateral sides of the central portion.
